(19) United States
(12) Patent Application Publication
(10) Pub. No.: US 2009/0280531 A1
(43) Pub. Date: Nov. 12, 2009

(54) Preparation of Soluble Capsid Proteins of Picornaviruses Using SUMO Fusion Technology

(75) Inventor: Ting-Fang Wang, Taipei (TW)

Correspondence Address: OCCHIUTI ROHLICEK & TSAO, LLP, 10 FAWCETT STREET, CAMBRIDGE, MA, 02138, US

(73) Assignee: Academia Sinica, NanKang (TW)

(21) Appl. No.: 12/436,215

(22) Filed: May 6, 2009

Related U.S. Application Data
(60) Provisional application No. 61/050,665, filed on May 6, 2008.

(51) Int. Cl. C12P 21/00; C07H 21/04

(52) U.S. Cl. 435/69.1; 536/23.1

(57) ABSTRACT

A method of producing a soluble capsid protein of a picornavirus using a novel and efficient SUMO fusion protein expression system.

### RELATED APPLICATION

This application claims priority to U.S. Provisional Application No. 61/050,665, filed on May 6, 2008, the content of which is hereby incorporated by reference in its entirety.

### BACKGROUND OF THE INVENTION

Picornavirus is a group of small animal viruses that invade the vertebrate intestinal tract or the central nervous system. Examples of picornavirus include hand-foot-and-mouth disease virus (HFMDV) and foot-and-mouth-disease virus (FMDV).

Infection of EV71, a strain of HFMDV, results in serious clinical manifestations, some of which are life threatening. See Ho et al., J. Microbiol. Immunol Infect 33:205-216 (2000). FMDV infection causes foot-and-mouth disease, which is one of the most contagious diseases in domestic cattle and swine. Davies, Res Vet Sci 73:195-199 (2000).

Currently, vaccination is the best approach for preventing and treating picornavirus infection. Capsid proteins of picornavirus, which constitute the protein shell of the virus, are desirable candidates for preparing anti-picornavirus vaccines. However, capsid proteins produced via recombinant technology are usually insoluble, thereby hindering their application as vaccine candidates. It is highly desired to develop a new method for preparing soluble capsid proteins of picornaviruses.

### SUMMARY OF THE INVENTION

The present invention provides a new method of preparing soluble picornavirus capsid proteins using a simple and efficient SUMO fusion protein expression system.

Accordingly, one aspect of this invention provides an expression construct containing a first nucleotide sequence encoding a Smt3 protein, and a second nucleotide sequence, which or a portion of which encodes a capsid protein of a picornavirus (e.g., a hand-foot-and-mouth disease virus or a foot-and-mouth-disease virus). The 3′ end of the first nucleotide sequence, replaced with a Sfo I restriction site, is linked to the 5′ end of the second nucleotide sequence via the Sfo I restriction site. The second nucleotide sequence can have a 5′ end Gly codon (e.g., GGC) linked directly to a capsid protein coding sequence, which preferably has the start codon ATG at its 5′ end. When introduced into a host cell, this expression construct expresses therein a fusion protein containing, from the N-terminus to the C-terminus, the Smt3 protein and the capsid protein. Cleavage of this fusion protein by U1p 1 protease produces the capsid protein having the exact amino acid sequence encoded by its coding sequence.

A “capsid protein” is a polypeptide that constitutes the protein shell of a picornavirus, e.g., VP1 of a EV71 HFMDV (EV71-VP1) or VP3 of a FMDV (FMDV-VP3). The term “restriction site” used herein refers to a nucleotide sequence recognizable by a restriction enzyme, or a nucleotide sequence generated by digestion of a restriction enzyme. The term “U1p1 protease” used herein refers to a polypeptide having the protease activity of Saccharomyces cerevisiae U1p1 protease. It can be a full-length Saccharomyces cerevisiae U1p1 protease or a fragment thereof (e.g., residues 403-621) that possesses protease activity, or a fusion protein containing the full-length protease or a fragment thereof and a protein tag (e.g., a His-tag).

Preferably, the expression construct described above further includes a third nucleotide sequence encoding a protein tag, the 3′ end of which is linked to the 5′ end of the first nucleotide sequence. This expression construct expresses in a host cell a fusion protein containing, from the N-terminus to the C-terminus, the protein tag, the Smt3 protein, and the capsid protein. Exemplary protein tags include, but are not limited to, hexa-His, Maltose binding protein, N-utilizing substance A, Thioredoxin (Trx), Calmodulin-binding protein, Glutathione S-transferase, and α-factor.

In another aspect, this invention provides a method of producing a soluble capsid protein of a picornavirus by (i) introducing any of the expression constructs described above into a host cell, (ii) producing in the host cell a fusion protein containing, from the C-terminus to the N-terminus, a capsid protein of a picornavirus, Smt3, and preferably, a protein tag, (iii) isolating the fusion protein from the host cell, and (iv) cleaving the fusion protein by a U1p1 protease to produce the capsid protein having the exact amino acid sequence encoded by the capsid protein gene contained in the expression construct.

The details of one or more embodiments of the invention are set forth in the description below. Other features or advantages of the present invention will be apparent from the following drawings and detailed description of several embodiments, and also from the appending claims.

### BRIEF DESCRIPTION OF THE DRAWINGS

The drawings are first described.

FIG. 1 is a diagram showing the process of generating expression vectors used in a new SUMO fusion protein expression system for producing soluble picornavirus capsid proteins.

FIG. 2 is a photograph showing fusion proteins His₆-Smt3-Rad51, His₆-Smt3-EV71-VP1, and His₆-Smt3-FMDV-VP3 expressed from expression constructs pHD-Rad51, pHD-EV71-VP1, and pHD-FMDV-VP3 in SDS-PAGE gels stained with Coomassie-blue. N: whole cell lysates derived from uninduced cells; I: whole cell lysate derived from induced cells; and S: soluble proteins derived from induced cells.

### DETAILED DESCRIPTION OF THE INVENTION

Capsid proteins of picornaviruses, e.g., EV71-VP1 and FMDV-VP3, are known to be poorly expressed in E. coli using conventional recombinant technology. See Van Komen et al., Methods in Enzymol. 408:445-462 (2006). Described herein is a method of producing soluble picornavirus capsid proteins in a simple and efficient SUMO fusion protein expression system. This system utilizes an expression vector containing a Smt3-encoding nucleotide sequence, which has its 3′ end replaced with a Sfo I restriction site. A Smt3 protein can be the Saccharomyces cerevisiae Smt3 protein, the amino acid sequence of which is shown below:

Amino acid sequence of Saccharomyces cerevisiae
Smt3
MSDSEVNQEAKPEVKPEVKPETHINLKVSDGSSEIFFKIKKTTPLRRLM
EAFAKRQGKEMDSLRFLYDGIRIQADQTPEDLDMEDNDIIEAHREQIGG

A Smt3 protein can also be a functional variant of the yeast Smt3 mentioned above, which is a C polypeptide that shares a high sequence homology with Smt3 (e.g., sequence identity at least 85%, 90%, 95%, 98%, or 99%). When fused with a target protein, a functional variant of yeast Smt3 can be cleaved by U1p1 protease to generate a free Smt3 protein having the mature C-terminus of yeast Smt3, i.e., -Gly-Gly, at the C-terminus of the free Smt3 protein. See Mossessova et al., Mol. Cell. 5:865-876 (2000).

In another example, a Smt3 protein is a fusion protein containing yeast Smt3 or its functional variant and a small protein tag, e.g., a hexa-His tag. The amino acid sequence of a His-tag fused yeast Smt3 protein is shown below:

Amino acid sequence of His-tag-Saccharomyces
cerevisiae Smt3 fusion protein:
MGSSHHHHHHSSGLVPRGSASMSDSEVNQEAKPEVKPEVKPETHINLKVS
DGSSEIFFKIKKTTPLRRLMEAFAKRQGKEMDSLRFLYDGIRIQADQTPE
DLDMEDNDIIEAHREQIGG

To prepare the expression vector of this invention, the 3′ end of the nucleotide sequence that encodes a Smt3 protein is replaced with a Sfo I restriction site for cloning downstream thereof a DNA fragment that encodes a capsid protein. In one example, the 3′ end of the Smt3 coding sequence, i.e., GGTGGT (encoding Gly-Gly), is replaced with a Sfo I site GGCGCC (encoding Gly-Ala). In another example, the GGTGGT sequence is replaced with GGTGGCGCC (encoding Gly-Gly-Ala). Preferably, this expression vector also includes a nucleotide sequence encoding a protein tag (e.g., His-tag) linked to the 5′end of the Smt3-encoding nucleotide sequence.

The term “expression vector” used herein refers to a plasmid containing, among other elements, a highly active promoter and one or more cloning sites downstream of the promoter.

This plasmid is used to introduce into and express in a host cell a target gene inserted into the plasmid via the cloning sites. Insertion of a DNA fragment encoding a protein of interest generates an expression construct.

The DNA fragment described above, coding for a picornavirus capsid protein, is then cloned into the expression vector described herein to form an expression construct capable of expressing in a host cell a Smt3-capsid fusion protein, or preferably, a protein tag-Smt3-capsid fusion protein. Such a DNA fragment can be prepared by conventional methods. Preferably, the DNA fragment is produced by sticky-end PCR such that it can be inserted into the expression vector mentioned above without being digested by restriction enzymes. After inserting the DNA fragment into the expression vector, the junction region of the Smt3 coding sequence and the DNA fragment can have the sequence of GGCGGCATG (encoding -Gly-Gly-M), in which ATG is the start codon of the capsid protein encoded by the DNA fragment. In one example, the capsid protein is VP1 of HFMDV EV71, the amino acid sequence of which is shown below:

Amino Acid Sequence of HFMDV-VP1:
GDRVADVIESSIGNSVSRALTQALPAPTGQNTQVSSHRLDTGEVPALQAA
EVGASSNTSDESMIETRCVLNSHSTAETTLDSFFSRAGLVGEIDLPLEGT
TNPNGYANWDIDITGYAQMRRKVELFTYMRFDAEFTFVACTPTGQVVPQL
LQYMFVPPGAPKPESRESLAWQTATNPSVFVKLTDPPAQVSVPFMSPASA
YQWFYDGYPTFGEHKQEKDLEYGACPNNMMGTFSVRTVGSLKSKYPLVVR
IYMRMKHVRAWIPRPMRNQNYLFKANPNYAGNSIKPTGTSRTAITTL

In another example, the capsid protein is VP3 of FMDV, the amino acid sequence of which is shown below:

Amino Acid Sequence of FMDV-VP3:
GIFPVACSDGYGGLVTTDPKTADPVYGKVFNPPRNLLPGRFTNLLDVAEA
CPTFLHFDGDVPYVTTKTDSDRVLAQFDLSLAAKHMSNTFLAGLAQYYTQ
YSGTINLHFMFTGPTDAKARYMVAYAPPGMEPPKTPEAAAHCIHAEWDTG
LNSKFTFSIPYLSAADYAYTASDVAETTNVQGWVCLFQITHGKADGDALV
VLASAGKDFDLRLPVDARTQ

The expression construct mentioned above is introduced into a host cell via methods known in the art to express the Smt3-capsid fusion protein or the protein tag-Smt3-capsid fusion protein. Any of such fusion proteins can be purified by, e.g., affinity column, and then cleaved by a U1p1 protease to yield the capsid protein having the exact amino acid sequence encoded by its coding sequence. Alternatively, U1p1 protease cleavage can be performed when the fusion protein is still bound to the affinity column.

Several embodiments of this invention are described in the following examples and also in Lee et al, Protein Science, 17(7):1241-1248 (2008).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Example 1

### Production of Fusion Proteins Containing Smt3 and Picornaviruses Capsid Proteins

The Saccharomyces cerevisiae Smt3 gene was cloned into pET32-Xa/LIC vector (Novagen, USA), downstream of the His₆-tag contained in this vector to produce a His₆-Smt3 expression vector.

The open reading frame of the Escherichia coli RecA protein was first cloned into the pET32-Xa/LIC vector (Novagen, USA) to generate a thioredoxin (Trx)-RecA expression construct. The DNA fragment encoding the Trx protein was then replaced by that of the His₆-Smt3 protein. As a result, the open reading frame of the Escherichia coli RecA protein is downstream of and in-frame with the His₆-Smt3 gene to produce a SUMO-RecA expression construct (pSUMO-RecA).

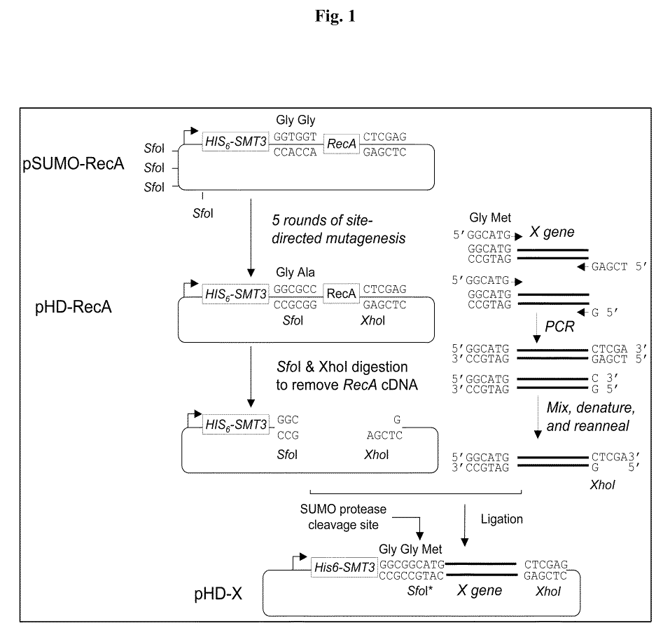

The pSUMO-RecA construct described above was then subjected to five rounds of site-directed mutagenesis reactions to mutate the four Sfo1 (5′GGCGCC3′) restriction sites in the backbone of pET32-Xa/LIC to either 5′GGCTCC3′ or 5′GGCACC3′, and to create a new Sfo1 restriction site at the SUMO protease cleavage site by mutating “GGTGGT,” encoding the two C-terminal residues ‘GlyGly’ of Smt3 to “GGCGCC”, encoding ‘GlyAla’. Next, the mutated pSUMO-RecA vector thus produced was subjected to Sfo1 and XhoI digestion to remove the DNA fragment encoding RecA, resulting in a linear vector pHD, one end of which is a Sfo1 site and the other end of which is a XhoI site. See FIG. 1. DNA fragments encoding EV71-VP1 and FMDV-VP3, prepared by sticky-end PCR (see Shih et al. Protein Sci. 11:1714-1719, 2002), were then inserted into vector pHD to produce expression constructs pHD-VP1 and pHD-VP3, in which a new “GlyGly” SUMO cleavage site is generated right before the first amino acid codon of the VP1 or VP3 gene (see FIG. 1).

The two expression constructs were then transformed into JM109(DE3)-competent cells, which were cultured overnight at 37° C. in the presence of 100 mg/L ampicillin to produce an overnight culture (15 mL). The overnight culture was then transferred to 1 L fresh Luria-Bertani medium, grew at 37° C. until it reached an OD₆₀₀ value of about 0.5-0.6, and IPTG (1 mM) was then added to the E. coli culture to induce protein expression. The induced cells were grown at 20° C. for 12 h, harvested, and then centrifuged at 9,000×g for 30 min. The cell pellet thus obtained were lyzed according to the method described in Wang et al., J. Biol. Chem. 268:26049-26051 (1993), except that a different lysis buffer [50 mM Tris-HCl (pH 7.4), 300 mM NaCl, 0.2 mM EGTA (pH 8.0)] was used here to prevent non-specific association of His₆-Smt3-VP1 or His₆-Smt3-VP3 with bacterial DNA. After centrifugation, the soluble fraction thus obtained was mixed with 2 mL of Ni²⁺ resins (Amersham, USA) to which the His₆-Smt3-VP1 and His₆-Smt3-VP3 fusion proteins binds. The Ni²⁺ resins were washed three times with 30 mL of wash buffer [50 mM Tris-HCl (pH 7.4), 300 mM NaCl, 0.2 mM EGTA (pH 8.0), 40 mM imidazole (pH 8.0)] and the fusion protein bound to them were then eluted.

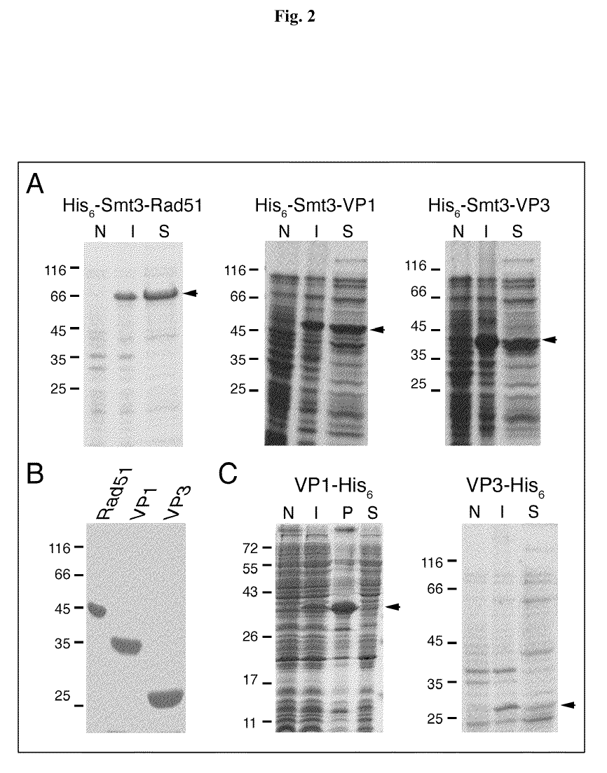

EV71-VP1 and FMDV-VP3, when expressed as fusion proteins with a His-tag only, were insoluble. See FIG. 2, panel C. When expressed in the SUMO fusion system described herein, both His₆-Smt3-VP1 and His₆-Smt3-VP3 proteins were soluble. See FIG. 4, panel A. Authentic VP1 and VP3 proteins were produced after cleavage of the fusion proteins by His₆-U1p1₄₀₃₋₆₂₁-His₆. See FIG. 2, Panel B. Edman degradation analysis confirmed that the N-termini of purified HFDV-VP1 and FMDV-VP3 were identical to those of the native HFDV-VP1 and FMDV-VP3. Mass spectrometry analysis revealed that the molecular weights of HFMDV-VP1 and FMDV-VP3 were 32,829 Da and 23,816 Da, respectively. The predicted molecular weights of these two proteins are 32,744 Da and 23,817 Da.

### Example 2

### Preparation of Free EV71-VP1 and FMDV-VP3 Proteins Via A One-Column Approach

Described below is a one-column approach to produce free EV71-VP1 and FMDV-VP3 capsid proteins from the His₆-Smt3-VP1 and His₆-Smt3-VP3 fusion proteins produced in Example 1 by U1p1 cleavage.

U1p1₄₀₃₋₆₂₁, a fragment of Saccharomyces cerevisiae U1p1 protein (amino acid residues 403-621), has been shown to cleave a C-terminal tagged yeast Smt3 in vitro, producing its mature form (i.e., C-terminal “Gly-Gly). See Mossessova et al., Mol. Cell. 5:865-876 (2000). The open reading frame of U1p1₄₀₃₋₆₂₁ was cloned into the pET28a vector (Novagen, USA) to generate an expression vector, which was then transformed into E. coli cells to express a His₆-U1p1₄₀₃₋₆₂₁-His₆ fusion protein. This recombinant enzyme, soluble in water, was purified from the crude extract of the transformed E. coli cells, using Ni²⁺ resins. The final yield was ˜20 mg/L Escherichia coli culture. The protein migrated as a single band on an SDS-PAGE gel stained with Coomassie blue, and with >99% purity as determined by densitometry. The His₆-U1p1₄₀₃₋₆₂₁-His₆ fusion protein exhibited a high affinity to Ni²⁺-resin and did not release from Ni²⁺-resins unless more than 300 mM imidazole or 100 mM EDTA was added to an elution buffer.

An E. coli crude extract containing the His₆-Smt3-VP1 or His₆-Smt3-VP3 fusion protein described in Example 1 was loaded to a column containing Ni²⁺-resins, which were then washed three times with 30 mL of the wash buffer also described in Example 1. Without elution, the Ni²⁺-column, bound with the His₆-Smt3-VP1 or His₆-Smt3-VP3 fusion protein, was then loaded with His₆-U1p1₄₀₃₋₆₂₁-His₆ to allow proteolytic cleavage of the His₆-Smt3-VP11VP3 fusion protein. The free VP1VP3 protein thus generated was then eluted from the Ni²⁺-resins. The final yield was 10 mg proteins per liter of cell culture.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An expression construct, comprising:
a first nucleotide sequence encoding a Smt3 protein, the 3′ end of the first nucleotide sequence being replaced with a Sfo I restriction site, and
a second nucleotide sequence, at least a part of which encodes a capsid protein of a picornavirus, the second nucleotide sequence being linked to the first nucleotide sequence via the Sfo I restriction site;
wherein the expression construct expresses a fusion protein containing, from the N-terminus to the C-terminus, the Smt3 protein and the capsid protein and cleaving the fusion protein by U1p1 protease produces the capsid protein.

2. The expression construct of claim 1, further comprising a third nucleotide sequence encoding a protein tag, wherein the expression vector expresses a fusion protein containing, from the N-terminus to the C-terminus, the protein tag, the Smt3 protein, and the capsid protein.

3. The expression construct of claim 1, wherein the second nucleotide sequence has a 5′ end Gly codon linked directly to a nucleotide sequence encoding the capsid protein.

4. The expression construct of claim 3, wherein the second nucleotide sequence has a 5′ end sequence GGCATG, in which GGC is the Gly codon and ATG is the start codon of the capsid protein.

5. The expression construct of claim 1, wherein the picornavirus is a hand-foot-and-mouth disease virus (HFMDV).

6. The expression construct of claim 5, wherein the hand-foot-and-mouth disease virus is EV71.

7. The expression construct of claim 6, wherein the capsid protein is HFMDV-VP1.

8. The expression construct of claim 1, wherein the picornavirus is a foot-and-mouth disease virus (FMDV).

9. The expression construct of claim 8, wherein the capsid protein is FMDV-VP3.

10. The expression construct of claim 2, wherein the protein tag is selected from the group consisting of hexa-His, Maltose binding protein, N-utilizing substance A, Thioredoxin, Calmodulin-binding protein, Glutathione S-transferase, and α-factor.

11. The expression construct of claim 10, wherein the protein tag is hexa-His.

12. The expression construct of claim 11, wherein the capsid protein is HFMDV-VP1 or FMDV-VP3.

13. A method of producing a capsid protein of a picornavirus, comprising:
providing an expression construct of claim 1,
introducing the expression construct into a host cell,
producing in the host cell a fusion protein containing, from the N-terminus to the C-terminus, the Smt3 protein and the capsid protein,
isolating the fusion protein from the host cell, and
cleaving the fusion protein by U1p1 protease to produce the capsid protein.

14. The method of claim 13, wherein the expression construct further contains a third nucleotide sequence encoding a protein tag and produces a fusion protein including, from the N-terminus to the C-terminus, the protein tag, the Smt3 protein, and the capsid protein.

15. The method of claim 13, wherein the second nucleotide sequence has a 5′ end Gly codon linked directly to a nucleotide sequence encoding the capsid protein.

16. The method of claim 15, wherein the second nucleotide sequence has a 5′ end sequence GGCATG, in which GGC is the Gly codon and ATG is the start codon of the capsid protein.

17. The method of claim 13, wherein the picornavirus is a hand-foot-and-mouth disease virus (HFMDV).

18. The method of claim 17, wherein the capsid protein is HFMDV-VP1.

19. The method of claim 13, wherein the picornavirus is a foot-and-mouth disease virus (FMDV).

20. The method of claim 19, wherein the capsid protein is FMDV-VP3.

21. The method of claim 13, wherein the protein tag is selected from the group consisting of hexa-His, Maltose binding protein, N-utilizing substance A, Thioredoxin, Calmodulin-binding protein, Glutathione S-transferase, and α-factor.

22. The method of claim 21, wherein the protein tag is hexa-His.


SEQUENCE LISTING (4 sequences)

SEQ ID NO: 1 - protein, 98 aa, Saccharomyces cerevisiae
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 2 - protein, 119 aa, Saccharomyces cerevisiae
MGSSHHHHHHSSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 3 - protein, 297 aa, Hand-foot-and-mouth disease virus
GDRVADVIESSIGNSVSRALTQALPAPTGQNTQVSSHRLDTGEVPALQAAEVGASSNTSDESMIETRCVLNSHSTAETTLDSFFSRAGLVGEIDLPLEGTTNPNGYANWDIDITGYAQMRRKVELFTYMRFDAEFTFVACTPTGQVVPQLLQYMFVPPGAPKPESRESLAWQTATNPSVFVKLTDPPAQVSVPFMSPASAYQWFYDGYPTFGEHKQEKDLEYGACPNNMMGTFSVRTVGSLKSKYPLVVRIYMRMKHVRAWIPRPMRNQNYLFKANPNYAGNSIKPTGTSRTAITTL

SEQ ID NO: 4 - protein, 220 aa, Foot-and-mouth disease virus
GIFPVACSDGYGGLVTTDPKTADPVYGKVFNPPRNLLPGRFTNLLDVAEACPTFLHFDGDVPYVTTKTDSDRVLAQFDLSLAAKHMSNTFLAGLAQYYTQYSGTINLHFMFTGPTDAKARYMVAYAPPGMEPPKTPEAAAHCIHAEWDTGLNSKFTFSIPYLSAADYAYTASDVAETTNVQGWVCLFQITHGKADGDALVVLASAGKDFDLRLPVDARTQ